(19) United States
(12) Patent Application Publication
(10) Pub. No.: US 2017/0296199 A1
(43) Pub. Date: Oct. 19, 2017

(54) DISPOSABLE, MODULAR SURGICAL INSTRUMENT

(71) Applicant: Aesculap AG, Tuttlingen (DE)

(72) Inventors: Jens Beger, Tuttlingen (DE); Tobias Nadler, Rottenburg (DE)

(73) Assignee: Aesculap AG, Tuttlingen (DE)

(21) Appl. No.: 15/511,960

(22) PCT Filed: Sep. 14, 2015
(86) PCT No.: PCT/EP2015/070969

§ 371 Date: Mar. 16, 2017

(30) Foreign Application Priority Data
Sep. 22, 2014 (DE) 10 2014 113 634.9

(51) Int. Cl. A61B 17/16; A61B 17/00

(52) U.S. Cl. CPC A61B 17/1611; A61B 2017/00469; A61B 2017/00464; A61B 2017/0023; A61B 2017/0042; A61B 2017/00367; A61B 2017/00424

(57) ABSTRACT

A disposable surgical instrument includes an instrument grip forming a receiving housing. A separate handle is mounted in the instrument grip in the manner of a rocker. A shaft tool includes a shaft having at least two relatively axially movable bar or rod components and an instrument head having patient engagement elements which can be actuated by the bar or rod components, of which one bar or rod component is fixed at the proximal end thereof to the instrument grip and the other bar or rod component has an articulation point on which the handle is directly articulated in such way that a manually actuated rocker movement of the handle about the articulation point on the handle side is transformed directly into an axial movement of the other bar or rod component on the articulation point on the shaft side.

### RELATED APPLICATION(S)

This application is the United States National Phase under 35 U.S.C. §371 of International Application No. PCT/EP2015/070969, filed Sep. 14, 2015, which is related to and claims the benefit of priority of German Application No. DE 10 2014 113 634.9, filed Sep. 22, 2014. The contents of International Application No. PCT/EP2015/070969 and German Application No. DE 10 2014 113 634.9 are incorporated by reference herein in their entireties.

### FIELD

The present invention relates to a surgical manually operable instrument which has a modular design and, consequently, can be equipped with different surgical tools in accordance with the principle of a modular system. Said concept is especially suited for being designed as a disposable instrument.

### BACKGROUND

Surgical medicine offers a plurality of applications of so called “single use instruments” which, due to their single use, need not be subjected to any elaborate purification and/or sterilization process and the handling of which therefore is easy and safe for the patient. Decisive to the profitability of “single use instruments” of this type are minimum manufacturing costs as well as sufficient functionality which should not rank behind conventional reusable instruments.

Disposable surgical instruments have been known already in the state of the art. They consist substantially of a number of basic elements made from plastic material which are functionalized by metal or ceramic inserts at particular points. In this way, instruments having a comparatively simple mechanism can be manufactured at low price by mass-manufacturing the basic elements of low-priced plastic material preferably by making use of generally known injection molding techniques, wherein the areas of the basic elements that are especially loaded in surgical application are reinforced by metal or ceramic trimmings. In this way, the costs for material and manufacture can be kept low and the function of the instrument can be ensured at least for the intended single use.

In particular, the afore-described design and manufacturing concept is applied to knives/scalpels, pincers, scissors/forceps, clamps and similar quasi-static instruments comprising a simple mechanism. It has turned out in this context, however, that surgical instruments having a complicated mechanical interior appear to be less suited as “single-use instruments”, because the advantages of the use of low-priced plastic materials are secondary vis-à-vis the high mounting costs in complicated instruments.

### SUMMARY

In view of the foregoing analysis of the state of the art, it is the object of the present invention to provide a surgical instrument the profitable manufacture of which is less dependent on the complexity thereof.

This object is achieved by a “disposable” surgical instrument comprising the features described herein.

Consequently, the basic idea of the present invention provides the application and the combination of at least three basic design measures:
the use of the modular design according to which one single basic element (for example the instrument grip) may be equipped with different tools and/or actuators/handles/actuating elements so as to realize a plurality of different surgical instruments with minimum effort so-to-speak in accordance with the modular system known from automotive engineering;
the reduction of the number of required elements for realizing the instrument mechanism so as to restrict the assembly of the instrument to few assembling steps (e.g. by assigning plural functions to one element); and
the selection of the materials for the individual instrument components/elements and for the working processes resulting therefrom so that as many of the elements as possible may be mass-produced with preferably low precision and only few elements have to be produced as individually manufactured goods with high precision (e.g. increased use of exclusively a plastic injection molding process for one element).

Concretely speaking, a “disposable” surgical instrument according to the invention includes the following modular elements:
a so called “handheld” comprising an instrument grip, preferably in the form of a pistol grip, which forms a receiving housing and a handle formed separately therefrom, preferably a trigger, trigger tongue or operating lever, which is received at least in portions in the receiving housing (is enclosed in portions by the receiving housing), and
a shaft tool consisting of a (rigid or resiliently flexible) shaft of at least two relatively axially movable (telescopic) bar or rod components and an instrument head/jaw part preferably comprising instrument branches or similar patient engagement elements which can be manually actuated by the handle (inside the receiving housing) via the two relatively axially movable bar or rod components.

The instrument grip and, resp., the receiving housing formed by the same forms/includes on its (distal) end portion facing the patient a receiving hole or similar mounting means in/on which the shaft (the inner or outer bar or rod component) can be mounted/supported on the instrument grip so that the relative movability of the bar/rod components (axial displacement of the outer or inner bar/rod component) is maintained. At least one of the (two) bar or rod components of the shaft (the outer or inner bar/rod component) and the instrument grip have/form a respective articulation point (on the inner housing side) on which the handle (e.g. rocker-type actuating lever) can be directly articulated such that a manual pivoting/rocking movement of the handle about the articulation point on the instrument grip side is transformed directly into an axial movement of the at least one bar or rod component on the shaft-side articulation point.

Preferably, the articulation point between the at least one bar or rod component and the handle (hereinafter referred to as shaft-side articulation point) and, further preferred, also the articulation point between the instrument grip and the handle (hereinafter referred to as articulation point on the instrument grip side) is formed as a click hinge. Such click hinge may be formed, for example, in that on the at least one bar or rod component and the instrument grip, where necessary, a bolt or hinge pin is tightly arranged/formed and, further preferred, on the preferably rocker-shaped handle clamp-type (partly open) pivot or hinge eyes are arranged/formed. It is outlined in this context that the assignment of the hinge pin and the pivot eyes to said elements may also be reversed, as a matter of course.

Further preferred, the instrument grip and, where necessary, also the handle are made from plastic material, especially injection-molding plastic, wherein the pivot eyes and hinge pins preferably may be formed from one material piece with the handle and/or the grip. It is also possible, however, to manufacture at least the pivot pin on the instrument grip side separately of a metal, for example, and to press-fit the same in corresponding bores on the instrument grip and on the handle, respectively.

The tool shaft may be made from metal, ceramic and/or plastic material. In particular, each of the two relatively axially movable bar or rod components may consist of metal, plastic or ceramic, wherein also a material pairing of a metal bar with a plastic or ceramic bar is possible so as to reduce the sliding friction between the two components.

Preferably, a bar or rod component (as a solid profile) is telescoped into the other bar or rod component (as a tubular profile) in a relatively movable manner, wherein on the radially outer bar or rod component the shaft-side articulation point for the handle is arranged/formed preferably in the form of a hinge bolt or pin, whereas the inner bar or rod component on its proximal end has a fastening means (e.g. locks or bolting holes etc.) by means of which the inner bar or rod component can be axially fixed on/in the handle.

The tool head may be, for example, in the form of a bone punch of a known design, a forceps/rongeur tool, holding forceps or scissors having a pivoting branch.

Between the handle and the instrument grip a biasing spring, for example a leaf spring, may be arranged for urging the handle into the home position (non-actuated position) thereof in which the tool is opened. The biasing spring may be formed integrally with the handle or the instrument grip of the same material or it is pre-assembled on the handle or the instrument grip as an externally fabricated component, preferably made from steel, or is injection-molded with the corresponding element during manufacture of the handle or the grip.

It is of advantage when the instrument grip and also the handle, where necessary, are manufactured by injection molding. For this purpose, a semi-crystalline thermoplastic structural material, e.g. based on polyphthalamide (PPA, Grivory GV-4h and the like) may be used. Said glass fiber reinforced material excels by the following properties:
high stiffness and strength,
little impact on the characteristics by absorption of humidity,
little absorption of humidity and water,
good dimensional stability and little distortion,
good chemical resistance typical for polyamide
excellent surface finish,
efficient and low-priced manufacture.

Further preferred, the instrument head/jaw part is made from sheet steel. For this purpose, the sheet metal can be shaped as desired and subsequently punched by means of a so called progressive die. The advantage of this design primarily resides in the low manufacturing costs.

The inner bar or rod component may consist of metallic round stock which may preferably be eroded or laser-treated and subsequently hardened. The outer bar or rod component may equally consist of metallic tubing which is preferably eroded or laser-treated.

Eroding offers the advantage that by an appropriate device it enables plural parts to be manufactured simultaneously, which allows saving manufacturing costs. The advantage of laser technology consists in the fact that the set-up time and the processing time are reduced vis-à-vis eroding.

As afore-mentioned, it is advantageous to assign plural functions and actuating options, resp., to at least one of the (afore-mentioned) elements of the surgical instrument in accordance with the invention (multi-function/operating element). It is favorable, for example, when the instrument grip is configured, in addition to the pistol grip shape, with a (transversely extending) through opening or eye having a diameter which is adapted so that at least one actuating finger of an operating person, preferably the thumb, may be reached through. In this way, the instrument grip can be received as the one branch of scissors or holding forceps. It is of further advantage when also the handle includes two actuating portions or offers actuating options correlated with the actuating options of the instrument grip. In so far the handle may include an open (not looped) as well as continuous (over the entire length thereof projecting from the receiving housing) concave actuating portion or actuating edge which is provided as a contacting or engaging edge for the operating finger(s), wherein additionally a through opening (extending transversely to the actuating direction) (which is comparable to the through opening on the instrument grip) is formed or provided on the handle, with the through opening being dimensioned for (at least) one actuating finger of the operating person reaching through.

In this way, the surgical instrument may be hand-held like a pistol and may be actuated by means of the trigger finger or may be received and handled like scissors at the eyes/through openings thereof.

### BRIEF DESCRIPTION OF THE DRAWING FIGURES

Hereinafter the invention will be illustrated in detail by way of a preferred embodiment with reference to the accompanying figures.

FIG. 1 illustrates the principle of functioning of a manually actuatable surgical instrument of the “disposable design” according to the invention in accordance with a preferred embodiment of the invention,

FIG. 2a, 2b illustrate two variants of a shaft tool/instrument head in the case of a bone punch and a rongeur tool,

FIG. 3 illustrates two handheld elements, i.e. a pistol grip and an actuating lever, according to the preferred embodiment of the invention,

FIG. 4 exemplifies two variants (rongeur and bone punch) of an inner bar or rod component of a shaft tool according to the invention,

FIG. 5 exemplifies two variants (rongeur and bone punch) of an outer bar or rod component of a shaft tool according to the invention,

FIG. 6 illustrates the two variants of a surgical instrument according to a preferred embodiment of the present invention in the mounted state, and

FIG. 7 describes the assembly operation for manufacturing a surgical instrument according to the invention in four diagrams.

### DETAILED DESCRIPTION

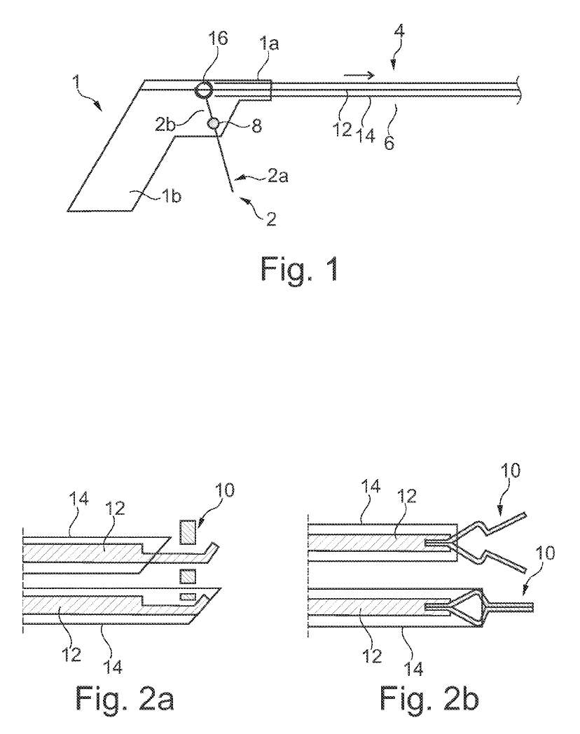

From FIGS. 1, 2a and 2b, a manually actuatable surgical instrument according to a preferred embodiment of the present invention is shown in a pictogram-like manner, with two variants for an optionally usable shaft tool being exemplified, i.e. a bone punch and a rongeur tool. It is outlined already in this context that also other shaft tools such as pincers, scissors or forceps may be chosen.

Accordingly, the surgical instrument according to the invention has a modular structure especially according to the modular principle and includes an instrument grip, preferably a pistol grip 1, which forms a receiving housing inter alia for an actuating lever 2 which is hinged in the manner of a rocker in the pistol grip 1 to an articulation point 8 on the handle side. A first actuating portion 2a of the rocker-type actuating lever 2 projects from the pistol grip 1 and, resp., from the receiving housing and serves as inserting/engaging area for a surgeon. A second force transmission portion 2b of the rocker-type actuating lever 2 on the side opposed to the actuating portion 2a with respect to the handle-side articulation point 8 serves as coupling transmission area for transferring the actuating force applied by a surgeon to a shaft tool 4.

The shaft tool 4 in the present case consists of a tool shaft 6 inserted in the pistol grip 1 and, resp., the receiving housing at its proximal end portion (facing away from the patient) and of an instrument head or jaw part 10 for the surgical effective engagement on the patient, as it is exemplified in FIGS. 2a, 2b in the form of the bone punch or the rongeur tool. In the present case, the tool shaft 6 is made of two bars 12, 14 telescoped into each other the inner bar 12 of which is fixedly held preferably in the form of a solid profile on the instrument grip 1 and, resp., in the receiving housing, whereas the outer bar 14 is supported to be axially movable in the receiving housing preferably in the form of a hollow profile (tube).

As one can infer already from FIG. 1, the outer bar 14 includes, preferably on its proximal end portion, a shaft-side articulation point 16 to which the actuating lever 2, especially the force transmission portion 2b thereof, is hinged on the end side. If, accordingly, the surgeon applies force to the actuating portion 2a of the actuating lever 2 in the direction of the pistol grip 1, the actuating lever 2 rocks/pivots in its central portion about the handle-side articulation point 8, while the actuating lever 2 pivots about the shaft-side articulation point 16 at the free end of the force transmission portion 2b and simultaneously advances the outer bar 14 relative to the inner bar 12 in the distally axial direction. Depending on the type of instrument head 10 the mechanism thereof is actuated in this way.

For example, FIG. 2a illustrates the schematic diagram of a bone punch as a variant of the instrument head 10. Accordingly, the distal end edge of the outer bar (bar tube) 14 may be in the form of a beveled cutting edge, whereas the distal end of the inner bar 12 may be shaped as a stop plate interacting with the cutting edge according to the anvil or bypass cutting principle, when the outer bar 14 is displaced relative to the inner bar 12 in the distal direction. As an alternative, in FIG. 2b, another variant of the instrument head 10 is shown in the form of a rongeur tool. The latter is generally made of two spring struts forming the opposed legs of forceps and being radially bulged in their longitudinal central portion. The proximal ends of the two legs are fixed on the distal end of the inner bar 12. When the outer tubular rod 14 is thus axially displaced in the distal direction, it sweeps over the radially bulged portions of the two legs and resiliently compresses the same due to the narrow cross-section of the tubular bar. In this way, the rongeur tool can be closed and opened again according to the principle of forceps.

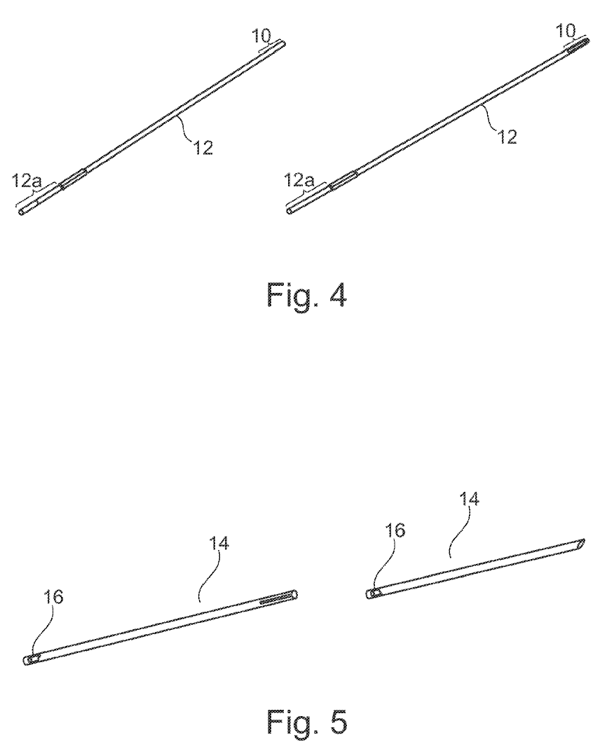

FIG. 4 shows a possible constructive assembly of the inner bar 12 according to the afore-mentioned two exemplary variants, whereas a possible constructive assembly of the outer bar 14 according to the afore-mentioned two exemplary variants is shown in FIG. 5.

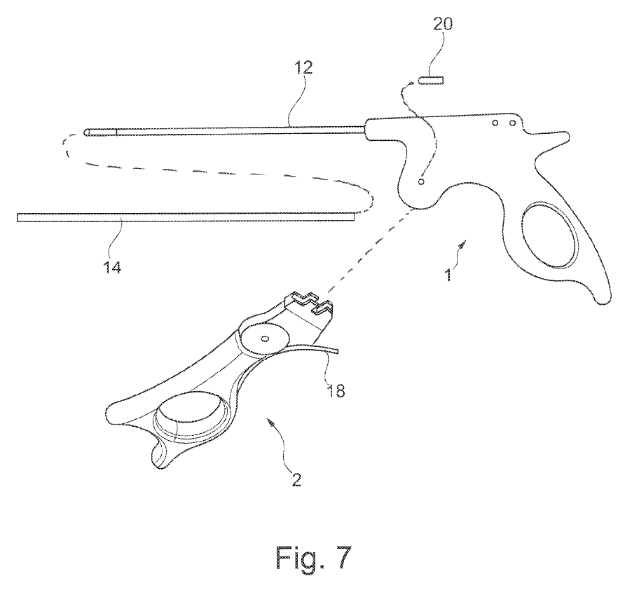

The surgical instrument according to the preferred embodiment of the present invention thus includes merely three main components for the modular system, i.e. the instrument grip 1 which is equal for all variants of the surgical instrument, the actuating lever 2 which optionally may have different lever ratios and the shaft tool 4 which is exchangeable for the different instrument variants. In addition, a biasing spring 18 (cf. FIG. 7), for example a leaf spring, may be provided which is disposed between the instrument grip 1 and the actuating lever 2 so as to bias the latter into the opening position of the shaft tool 4.

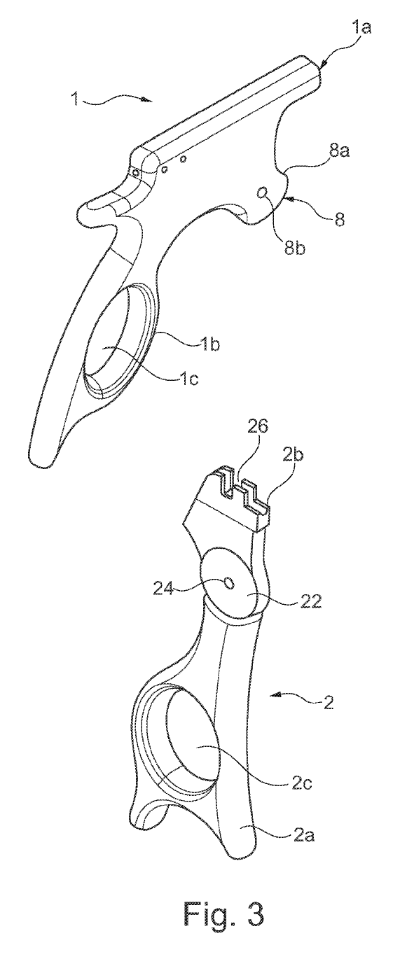

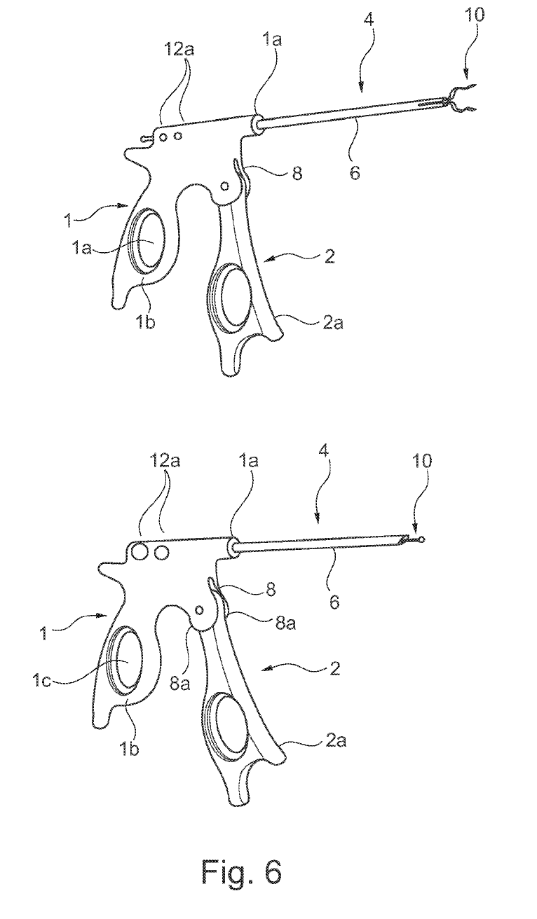

The instrument grip 1 and the actuating lever 2 are shown separately from each other in FIG. 3 and in the mounted state in FIG. 6.

Accordingly, in its distal area the instrument grip 1 forms sort of a receiving duct 1a into which the tool shaft 4 can be inserted and a holding area 1b having the contours of a pistol grip. Additionally, in the holding area 1b a through opening 1c corresponding to a scissors grip (for a thumb/finger reaching therethrough) is formed. Finally, in the distal area below the receiving shaft 1a the handle-side articulation point 8 is disposed. In the present case, it consists of a projection extending along the holding area 1b and being formed of two eye plates 8a spaced apart in parallel. Each of the eye plates 8a includes an aligned through bore 8b into which a hinge pin 20 can be inserted.

The actuating lever 2 equally includes in its first actuating portion 2a a rim contour (open/non-looped grip edge) which promotes the lever 2 being held by the fingers of a surgeon and, in addition, a through opening 2c corresponding to a scissors grip for a finger (thumb) reaching therethrough. In this respect, the instrument grip 1 including the actuating lever 2 mounted thereto may be optionally held and handled like a pistol grip or like a scissors grip so as to cope with different shaft tools 4.

The actuating lever 2 includes in its central portion a flattening 22 provided with a through hole 24. The flattening 22 is configured so as to be slidingly introduced between the two parallel eye plates 8a, wherein the subsequently inserted hinge pin 20 pivotally retains the actuating lever 2 on the instrument grip 1. As is further evident from FIG. 3, the actuating lever 2 forms a clamp eye or driving fork 26 at the distal end of its second force transmission portion 2b. Said clamp eye 26 is adapted to be pivotally engaged with the hinge pin (not shown in detail) of the shaft-side articulation point 16.

In FIGS. 4 and 5 the two variants of tool shafts 4 are exemplified for the bone punch and the rongeur tool as an engineering structural drawing.

Accordingly, the inner bar 12 of each tool shaft 4 according to FIG. 4 includes the distal end portion for receiving or forming the patient engagement element on the instrument head 10 as afore-described already and a proximal end portion which is formed as or provided with a fixing device 12a. Concretely speaking, the fixing device 12a may consist of one or two transverse bores (cf. especially FIG. 6) which is/are aligned with one or two transverse bores on the instrument grip 1 when the tool shaft 4 is completely inserted in the receiving duct la. Then screws or splints may be inserted into the transverse bores 12a so as to axially fix the inner bar 12 within the instrument grip 1.

The outer tubular bar 14 according to FIG. 5 basically has an inner diameter which enables the same to slide along the inner bar 12 in a guided manner. The outer tubular bar 14, too, forms or includes at its distal end a patient engagement element that interacts with the patient engagement element of the inner bar 12 while forming the instrument head or jaw part 10. In contrast to the inner bar 12, however, the outer bar 14 includes at its proximal end the shaft-side articulation point 16 which is a transverse bolt in the present case. The length and the diameter of the transverse bolt are adapted to the clamp eye 26 of the actuating lever 2 such that the latter can be assembled to the outer tubular bar 14 on the shaft-side articulation point 16 without the use of any tools.

The assembly of a surgical instrument according to the preferred embodiment of the present invention, presently in the form of the bone punch, is illustrated in FIG. 7.

Accordingly, at first, the inner bar 12 is inserted into the receiving duct 1a of the instrument grip 1 and is axially secured by means of two transverse splints. Then the outer tubular bar 14 is slipped over the inner bar 12 and is equally inserted into the receiving duct 1a until the latter proximally abuts at the end side on the front transverse splint.

As is evident from FIG. 7, a leaf spring 18 has already been pre-mounted on the actuating lever 2. This may be performed by riveting, screwing, bonding or casting when the actuating lever 2 is injection-molded. The actuating lever 2 prepared in this way is now inserted below the receiving duct 1a between the two parallel eye plates 8a into the instrument grip 1 until the latter locks/engages with its end-side clamp eyes 26 on the transverse bolt (articulation point 16) of the outer tubular bar 14 in a hinge-like manner. Finally a hinge pin 20 is put into the through bores 8b at the eye plates 8a and into the through bore 24 at the actuating lever 2 and in this way the actuating lever 2 is hinge-coupled to the instrument grip 1. In this position, the leaf spring 18 resiliently contacts the outside of the instrument grip 1 and thus biases the actuating lever 2 in the opening direction of the instrument.

As one can infer from the foregoing description, merely four assembly steps are required to complete the surgical instrument. Said steps can be carried out quickly and without any major manufacturing expenditure. Both the instrument grip 1 and the actuating lever 2 are made from injection-molded plastic and hence manufacture thereof is quite inexpensive. Merely the shaft tool 4 and the jaw part 10 are manufactured at least partly or completely of metal or ceramic. In so far the manufacturing costs in terms of material, manufacture of the modular elements and assembly thereof can be minimized. Besides, all elements can be assembled from outside of the instrument grip 1 without the use of any tools and are also accessible from outside. In the present case, this is no problem in view of sterilization/purification as well as susceptibility to damage, however, as the instrument is provided as a disposable instrument which will be disposed of after one single use.

Summing up, the invention discloses a disposable surgical instrument which, in accordance with the principle of a modular system, has the following elements:
an instrument grip 1, preferably a pistol grip, which forms a receiving housing in which instrument grip a separate handle such as an actuating lever 2 is mounted in the manner of a rocker on an articulation point 8 on the handle side, and
a shaft tool 4 consisting of a shaft 6 comprising at least two relatively axially movable bar or rod components 12, 14 and an instrument head 10 having patient engagement elements which can be actuated by the two relatively axially movable bar or rod components 12, 14 of which one bar or rod component 12 is fixed at the proximal end thereof to the instrument grip and the other movable bar or rod component 14 has a shaft-side articulation point 16 on which the handle 2 is directly articulated in such way that a manually actuated rocker movement of the handle 2 about the handle-side articulation point 8 is transformed directly into an axial movement of the other bar or rod component 14 on the shaft-side articulation point 16.

1. A disposable surgical instrument comprising:
an instrument grip which forms a receiving housing in which instrument grip a handle is mounted in the manner of a rocker on an articulation point on the handle side, and
a shaft tool comprising a shaft comprising at least two relatively axially movable bar or rod components and an instrument head or jaw part having patient engagement elements which can be actuated by the at least two relatively axially movable bar or rod components, of which one of the bar or rod components is fixed to the instrument grip and another of the bar or rod components has a shaft-side articulation point on which the handle is directly articulated in such way that a manually actuated pivoting/rocker movement of the handle about the handle-side articulation point is transformed directly into an axial movement of said another of the bar or rod components on the shaft-side articulation point,
said one of the bar or rod components is in the form of a solid profile and is relatively movably telescoped into said another of the bar or rod components in the form of a tubular profile,
said shaft-side articulation point is arranged at said another of the bar or rod components in the form of a hinge bolt or pin, and
said one of the bar or rod components at its proximal end has a fastening mechanism by which said another of the bar or rod components is fixed to or fixed within the instrument grip.

2. The surgical instrument according to claim 1, wherein at least one of the shaft-side articulation point and the handle-side articulation point forms a click hinge with the handle, while on the other said another of the bar or rod components and the instrument grip, a bolt or hinge pin is fixedly arranged or formed, and on the rocker-shaped handle clamp-type hinge eyes are arranged or formed.

3. The surgical instrument according to claim 1, wherein at least one of the instrument grip and the handle is made from plastic material.

4. The surgical instrument according to any claim 1, wherein each of the at least two relatively axially movable bar or rod components comprises metal, plastic or ceramic or in that a material pairing of a metal bar with a plastic or ceramic bar is provided.

5. (canceled)

6. The surgical instrument according to claim 1, wherein the instrument head is in the form of a bone punch, a forceps/rongeur tool, holding forceps or scissors having a pivoting branch.

7. The surgical instrument according to claim 1, wherein between the handle and the instrument grip a biasing spring is arranged for urging the handle the home position thereof in which the tool is opened.

8. The surgical instrument according to any claim 1, wherein the instrument grip is manufactured by injection molding.

9. The surgical instrument according to claim 8, wherein a semi-crystalline thermoplastic structural material is used.

10. The surgical instrument according to claim 1, wherein the instrument head is made from sheet steel.

11. The surgical instrument according to claim 1, wherein
the instrument grip includes a holding area which is shaped as a pistol grip for being encompassed in the palm of an operator's hand and with a through opening additionally introduced in the same or arranged thereon for reaching through an operating finger of an operating person, and
the handle has an open continuously concave actuating portion which is in the form of a non-looped contacting or engaging edge for the operating fingers having an additional through opening for at least one operating finger the operating person.
